(19) United States
(12) Patent Application Publication
(10) Pub. No.: US 2012/0015013 A1
(43) Pub. Date: Jan. 19, 2012

(54) MATRIX CONTAINING METAL OXIDE PARTICLES AND USE OF SAME IN COSMETIC COMPOSITIONS

(75) Inventors: DAVID SCHLOSSMAN, South Plainfield, NJ (US); Yun Shao, South Plainfield, NJ (US); Pascal Delrieu, South Plainfield, NJ (US)

(73) Assignee: Kobo Products, Inc., South Plainfield, NJ (US)

(21) Appl. No.: 13/231,110

(22) Filed: Sep. 13, 2011

Related U.S. Application Data
(63) Continuation-in-part of application No. 12/420,983, filed on Apr. 9, 2009.

(51) Int. Cl. A61K 8/02; A61Q 17/04

(52) U.S. Cl. 424/401; 424/59

(57) ABSTRACT

A macroparticle sunscreen powder and methods of making the same. The macroparticle sunscreen powder includes a plurality of macroparticle particles that include UV attenuating particles in a matrix material selected from the group consisting of a natural latex, polyacrylate, polymethacrylate, polyurethane, polyvinyl acetate, styrene-butadiene rubber, acrylonitrile butadiene styrene copolymer, and combinations thereof.

### RELATED APPLICATION

This application is a continuation-in-part of U.S. Ser. No. 12/420,983 filed on Apr. 9, 2009.

### FIELD

The present invention relates to a matrix containing ultraviolet attenuating pigments, especially nano- or micro-metal oxide particles, methods for forming the matrix, and compositions containing the same.

### BACKGROUND

Light scattering pigments have long been used to provide protection from ultraviolet light. For many years, continuing to the present, ointments comprising large particles of titanium dioxide have been applied to the skin, typically seen as a white smear on the nose or cheeks. While consumer perception appears to be that such visually obvious applications confer a high degree of protection from ultraviolet radiation, it has long been known that much smaller zinc oxide and titanium dioxide particles suspended in a medium of, for example, oil, will not only attenuate ultraviolet light but will also appear transparent and thus invisible. This is achieved because the particles have a diameter substantially smaller than the wavelength of visible light.

Micro-zinc oxide was developed and has been used since the 1980s. JP 60-231607 disclosed the use of zinc oxide with a primary particle size of 1.0-60 nm. U.S. Pat. No. 5,032,390 disclosed the use of zinc oxide with a primary particle size of 70-300 nm. These zinc oxides are transparent on the skin and aesthetically appealing and are extensively used nowadays. These micro grades typically have primary particle sizes of less than 100 nm when analyzed, and are referred to as nano particles.

Concerns of skin penetration and bioaccumulation, however, have arisen regarding the use of particles with one or more dimensions less than 100 nm on the skin.

### SUMMARY

To address this concern, the challenge is to create particles that are larger yet still have the desirable attributes of good UV attenuation and transparency that the micro particles offer. Accordingly the aim of the composition herein is to create an overall larger particle size while maintaining the desirable characteristics of micro particles such as metal oxides like zinc oxide and titanium dioxide. In accordance with the invention, this has been accomplished by entrapping the micro particles in a matrix material that comprises a natural latex, polyacrylate, polymethacrylate, polyurethane, polyvinyl acetate, styrene-butadiene rubber, acrylonitril butadiene styrene copolymer, or combinations thereof. The matrix is dried and a fine powder is recovered. Surprisingly, the resulting dried powder had the same UV attenuating power as a dispersion of the same micro metal oxide. Additionally, the matrix material is such that after formation of the macroparticle the micro particles cannot diffuse out of the resulting macroparticles.

In one aspect, a method for forming a macroparticle powder capable of filtering ultraviolet radiation is disclosed. The method includes the steps of dispersing a UV shielding agent in water, adding a matrix material selected from the group consisting of a natural latex, aqueous emulsions or dispersions of polyacrylate, polymethacrylate, polyurethane, polyvinyl acetate, styrene-butadiene rubber, acrylonitrile butadiene styrene copolymer, and combinations thereof to the UV shielding agent in water to form a mixture, and spray drying the mixture to obtain macroparticles.

These macroparticles may be formulated into dispersions and/or into cosmetic compositions, including but not limited to, sunscreens or suntan lotions, bronzers, other lotions, gels, hairsprays, mascara, foundation, and face powder.

### DETAILED DESCRIPTION

A macroparticle powder that includes a matrix supporting a plurality of particles of UV attenuating agents, that is a sunscreen particle that, in a conventional sunscreen tends to be transparent to visible light, but at the same time tends to block ultraviolet light, has been developed that eliminates the discrete nano particles of the sunscreen particles. These UV attenuating agents include inorganic sunscreen particles such as titanium dioxide and zinc oxide that are considered nanoparticles and have an mean primary particle size of less than 100 nm. In addition to such inorganic sunscreen particles, the matrix may include organic materials having the characteristics of zinc oxide or titanium dioxide, combinations of inorganic sunscreens, and combinations of inorganic sunscreens with such polymeric materials. The polymeric materials include suitable organic UV attenuating agents such as, but not limited to, a triazine, an oxanilide, a triazole, a vinyl group-containing amide, a cinnamic acid amide, a sulfonated benzimidazole, other classes of substances known as UV attenuating or shielding agents, and combinations thereof.

The phrase “macroparticle powder,” as used herein, refers to the particles produced by dispersing UV shielding agents in a solid matrix material. In one embodiment, the macroparticle powder particles have a mean particle size of between about 0.2 μm to about 100 μm, and preferably from about 0.5 μm to about 50 μm.

The phrase “primary particle size,” as used herein, refers to the diameter of a particle when the particles are substantially spherical or spheroidal and the width or the largest dimension of a particle when the particles are acicular. Acicular shapes include, but are not limited to, nodular, acicular, granular, ellipsoidal, hexagonal, prismatic, star-like, flakes, and Y-shaped. For UV attenuating agents, the particle size is typically reported as the mean primary particle size as determined by electron microscopy. Frequently, the primary particles form aggregates due to inter-particle forces.

The phrase “mean particle size”, as used herein with respect to the particle size of the macroparticles, means the mean of the diameters for greater than 200 randomly selected macroparticles (because there could be thousands or only a few particles on the photograph; with too few, there would be an absence of statistical significance, but with too many, there would be too much labor to calculate; hence the threshold here was set at >200 particles to be measured and sizes averaged) measured with an electronic micrograph, or the mean size measured with a light scattering analyzer.

Examples of organic UV shielding nanoparticle particulates that may be useful in the present invention include those described in U.S. Pat. No. 5,869,030 to Dumler et al. and U.S. Pat. No. 6,495,122 to Fankhauser et al., the contents of these patents are hereby incorporated by reference.

The inorganic UV attenuating agents useful in the present invention are those typically used for shielding ultraviolet light. In accordance with certain embodiments of the present invention, the inorganic UV attenuating agents are metal oxides and more specifically metal oxides selected from TiO₂, ZnO, zirconium oxide, cerium oxide and any metal oxides or other materials that can absorb and/or scatter UV light while maintaining an acceptable degree of transparency, and mixtures thereof. The metal oxide particles may have a mean particle size of from about 1 nm to about 100 nm.

The UV attenuating agent particles, whether inorganic or organic, incorporated in the matrix can be uncoated or can be coated, for example, with a metal oxide or metal hydroxide, and/or organic compounds such as, but not limited to, fatty acids, metal soap, silicones, silanes, etc. The UV attenuating agent particles can be rendered hydrophilic or hydrophobic by the presence of a coating. Other suitable surface treatments may include: silica, phosphate esters (including lecithins), perfluoroalkyl alcohol phosphates, fluorosilanes, isopropyl titanium triisostearate, stearic or other fatty acids, silanes, dimethicone and related silicone polymers or combinations thereof. Methods of coating zinc oxide particles are known to one of skill in the art, as well as various other coatings and the amount of coating to be used.

For example, metal oxide particles may be coated with oxides of other elements such as oxides of aluminum, zirconium or silicon, or mixtures thereof such as alumina and silica as disclosed in GB-2205088-A, the teaching of which is incorporated herein by reference. Alternately, such powders may be treated with boron nitride or other known inorganic coatings, singly or in combinations.

The inorganic coating may be applied using techniques known in the art. One such process includes forming an aqueous dispersion of the UV attenuating micro particles in the presence of a soluble salt of the inorganic element whose oxide will form the coating. This dispersion is usually acidic or basic, depending upon the nature of the salt chosen, and precipitation of the inorganic oxide is achieved by adjusting the pH of the dispersion by the addition of acid or alkali, as appropriate. The inorganic coating, if present, is preferably applied as a first layer to the surface of the UV attenuating particles.

In another embodiment, these powders may include an organic coating that gives the UV attenuating particles hydrophobic properties. The organic coating may be applied to the inorganic coating, if present, or directly to the UV attenuating particles. The hydrophobic coating agent may be, for example, a silicone, a silane, a metal soap, a titanate, an organic wax, and mixtures thereof. The hydrophobic coating may alternatively include a fatty acid, for example, a fatty acid containing 10 to 20 carbon atoms, such as lauric acid, stearic acid, isostearic acid, and salts of these fatty acids. The fatty acid may be isopropyl titanium triisostearate. With respect to the silicone, the hydrophobic coating may be a methicone, a dimethicone, their copolymers or mixtures thereof. The silicone may also be an organosilicon compound, for example dimethylpolysiloxanes having a backbone of repeating —Me₂SiO— units (“Me” is methyl, CH₃), methyl hydrogen polysiloxanes having a backbone of repeating —MeHSiO— units and alkoxysilanes of formula RₙOSiH₍₄₋ₙ₎ where “R” is alkyl and “n” is the integer 1, 2 or 3. With respect to the silane, the hydrophobic coating agent may be an alkoxysilanes, for example, an alkyltriethoxy or an alkyltrimethoxy silanes available from OSI Specialities or PCR. The alkoxysilane may be a triethoxycaprylylsilane or a perfluoroalkylethyl triethoxysilane having a C3 to C12 alkyl group that is straight or branched. One such alkoxysilane is Dynasylan® OCTEO available from Degussa AG. With respect to the metal soap, the hydrophobic coating agent may be a metal myristate, metal stearate, a metal palmitate, a metal laurate or other fatty acid derivatives known to one of skill in the art. The metal, for example, may be magnesium or aluminum. With respect to the titanate, the hydrophobic coating agent may be an organotitanate as taught in U.S. Pat. No. 4,877,604 to Mitchell Schlossman (“Schlossman '604” hereinafter), the disclosure of which is herein incorporated by reference thereto. Schlossman '604 discloses isopropyl titanium triisostearate as one preferred coating agent. With respect to the organic wax, the hydrophobic coating agent may be a synthetic wax like polyethylene or a natural wax like carnauba wax.

If the UV attenuating particles are coated, then the substance forming the coating may be present in the composition in an amount in a range of about 0.1% to about 35% by weight of the resulting coated UV attenuating particles. In another embodiment, the coating may be 4% to 20% by weight of the resulting coated UV attenuating particles.

The macroparticle powder as described herein may contain a single type of UV attenuating agent or combination of UV attenuating agents. Furthermore, the UV attenuating agents in the macroparticle powder may also be combined with additional substances, such as, for example, photostabilizers, cosmetic oils and/or anti-oxidants.

The matrix material is one that is capable of forming, for example, a gel to entrap a plurality of particles of the UV attenuating agent or a material exhibiting sufficient adhesion to bind the UV attenuating agent particles without significantly interfering with the ultraviolet filtering ability of the UV attenuating agent particles or the transparency of the composition in the visible light region. The matrix material is one that when dried is insoluble and does not swell in water or oil. The matrix material is selected from a natural latex, aqueous emulsions or dispersions of polyacrylate, polymethacrylate, polyurethane, polyvinyl acetate, styrene-butadiene rubber, acrylonitril butadiene styrene copolymer, or combinations thereof. The matrix is spray dried after being mixed with the UV attenuating agent and a dried, fine powder is recovered. The resulting macroparticles are themselves a matrix having a plurality of the UV attenuating agents embedded therein. This matrix is generally similar to cement, for example, where there is a primary material in which lumps of coarser material, as of an aggregate, are embedded. The matrix material is not an absorbent material, powder, sphere, microsphere, or the like. Surprisingly, the resulting dried powder had the same UV attenuating power as a dispersion of the same micro metal oxide dispersed or milled in oil or water.

The macroparticle powder made by the process discussed above typically comprises the UV attenuating agent and matrix material present at a ratio (by dry weight) of about 5:1 to about 1:20, more preferably from about 2:1 to about 1:2. Each macroparticle powder particle typically contains a plurality of UV attenuating agent particles. It is also contemplated that various particle types and/or particles of various sizes may be combined in a single macroparticle powder particle.

Macroparticle powders may be formed by any method capable of producing the macroparticle powder particles at the appropriate size. The present invention is described in more detail by reference to spray drying to form the macroparticle powder. However, the present invention should not be considered limited to this process and other processes such as freeze drying, prilling, extrusion/spherization, emulsion/dispersion process and precipitation may also be used. The dried, fine powder resulting after macroparticle formation may have a particle size of about 0.2-100 μm.

It is also contemplated that after macroparticle formation, the macroparticles may be screened, milled, or reduced in size by other processes to assure proper particle size to meet a required size specification.

Spray drying is a particle processing technology that transforms a liquid feed stock into a powder product by first spraying the feed stock to create droplets, and then evaporating the feed stock liquid through the use of a heated drying medium, typically air. The liquid feed stock can take the form of a solution, suspension, liquid-paste or emulsion, and should be pumpable and capable of droplet formation. The feed stock composition in accordance with the present invention comprises the UV attenuating agent, the matrix material and a dispersion media, such as, for example, water or organic solvents.

The UV attenuating agent macroparticle powder prepared in accordance with the present invention may be formulated into cosmetic compositions, sunscreen compositions, or other compositions as needed to provide the desired ultraviolet filtering properties. The UV shielding agent macroparticle powder may be incorporated into the finished compositions with a concentration of UV shielding agent from about 1 to about 80% by weight, more preferably from about 2-20%, and most preferably from about 3 to about 10% by weight to provide an effective and typical protecting concentration of the ultraviolet shielding agent. The finished compositions may be in the form of suntan lotions, bronzers, other lotions, gels, hairsprays, mascara, foundation, face powder, aerosol foam creams or emulsions, and so forth.

The cosmetic compositions containing the macroparticle powders above may be formulated in various forms by conventional methods. Although the forms are not particularly limited, the cosmetics may be formulated as various makeup products as noted above and including lotions, emulsions, creams, ointments, aerosol cosmetics, powdery foundations, powdery eye shadows, emulsifying foundation creams, lipsticks, hair care preparations, and skin cleansers.

Generally, the macroparticle sunscreen powder functions, from an optical standpoint, in the same manner as the much smaller nanoparticles of the sunscreen, because the index of refraction of the matrix is substantially lower than the index of refraction of the sunscreen particles, whether the sunscreen particles be polymeric or metal oxides.

The present invention is described in more detail by the following non-limiting example.

### Example 1

Six (6) kg of a titanium dioxide suspension in water that has a TiO₂ present as 45% by dry weight thereof (available as W45AQ from Kobo Products, Inc.) was dispersed in 2 kg of water. Then 2 kg of an acrylate emulsion that is 50% resin by dry weight (available as Diatosol 500AD from Kobo Products, Inc.) was added to the dispersed TiO₂ with mixing. Thorough mixing was performed in a jacketed tank to ensure a uniform dispersion. The resulting mixture was then spray dried using a conventional spray drying apparatus at 375° C.

Particle size of the spray dried product was first assessed under an optical microscope. The matrix-TiO₂ powder was observed to have a mean particle size of 15 nm.

The UV attenuation provided by the matrix-TiO₂ powder was tested by comparison to a dispersion of the nanoparticle TiO₂ powder used in this Example in a sunscreen emulsion.

TABLE 1

| Sunscreen | TiO2 type | TiO2 (%) | SPF |
|---|---|---|---|
| A | spray dried macroparticles | 6.3 | 15.9 |
| | made in Example 1 | | |
| B | Kobo IN60TS (15 nm | 6.4 | 16.0 |
| | TiO2 dispersion in ester) | | |
| C | MT-100T (15 nm TiO2 | 6.4 | 9.6 |
| | powder) | | |
| D | MT-100Z(15 nm TiO2 | 6.0 | 11.1 |
| | powder) | | |

What is claimed:

1. A macroparticle sunscreen powder comprising:
a plurality of macroparticle particles, the macroparticle particles comprising UV attenuating particles in a matrix material selected from the group consisting of a natural latex, polyacrylate, polymethacrylate, polyurethane, polyvinyl acetate, styrene-butadiene rubber, acrylonitrile butadiene styrene copolymer, and combinations thereof.

2. The macroparticle sunscreen powder of claim 1, wherein said matrix material is transparent or translucent.

3. The macroparticle sunscreen powder of claim 1, wherein, said macroparticle particles have an mean particle size of from about 0.2 μm to about 100 μm, and said UV attenuating particles have an mean primary particle size of less than 100 nm.

4. The macroparticle sunscreen powder of claim 3, wherein the macroparticle particles have generally the same UV attenuating power as a dispersion in water or oil of the UV attenuating particles itself.

5. The macroparticle sunscreen powder of claim 1, wherein the UV attenuating particles comprise inorganic UV attenuating particles.

6. The macroparticle sunscreen powder of claim 5, wherein the inorganic UV attenuating particles comprise metal oxide particles selected from the group consisting of TiO₂, ZnO, and combinations thereof.

7. The macroparticle sunscreen powder of claim 6, wherein said metal oxide particles have an mean particle size of less than 100 nm.

8. The macroparticle sunscreen powder of claim 1, wherein the macroparticle particles comprise a ratio of UV attenuating particles to matrix material of about 5:1 to about 1:20 by dry weight.

9. The macroparticle sunscreen powder of claim 1 wherein the macroparticle particles comprise a ratio of UV attenuating particles to matrix material of about 2:1 to about 1:2 by dry weight.

10. A dispersion incorporating the macroparticle sunscreen powder of claim 1.

11. A cosmetic composition incorporating the macroparticle sunscreen powder of claim 1.

12. A method for forming a macroparticle powder capable of filtering ultraviolet radiation comprising:
dispersing a UV shielding agent in water;
adding a matrix material selected from the group consisting of a natural latex, aqueous emulsions or dispersion of polyacrylate, polymethacrylate, polyurethane, polyvinyl acetate, styrene-butadiene rubber, acrylonitrile butadiene styrene copolymer, and combinations thereof to the UV shielding agent in water to form a mixture; and
drying the mixture to obtain macroparticles.

13. The method of claim 12, wherein the macroparticles have an average particle size of about 3-100 μm.

14. The method of claim 12, wherein the UV shielding agent is selected from the group consisting of TiO₂, ZnO and combinations thereof.

15. The method of claim 14, wherein the UV shielding agent has a mean particle size of less than 100 nm.

16. The method of claim 12, wherein drying includes spray drying.

17. The method of claim 12, wherein the UV attenuating agent to matrix material comprise a ratio of about 5:1 to about 1:20 by dry weight.

18. The method of claim 12, wherein the UV attenuating agent to matrix material comprise a ratio of about 2:1 to about 1:2 by dry weight.
